(19) United States
(12) Patent Application Publication
(10) Pub. No.: US 2003/0171371 A1
(43) Pub. Date: Sep. 11, 2003

(54) Immunosuppressant n-acyles homoserine lactones

(76) Inventors: Barrie Walsham Bycroft, Nottingham (GB); David Idris Pritchard, Leicester (GB); Siri Ram Chhabra, Leicester (GB); Doreen Hooi, Nottingham (GB)

Correspondence Address: BOZICEVIC, FIELD & FRANCIS LLP, 200 MIDDLEFIELD RD, SUITE 200, MENLO PARK, CA, 94025, US

(21) Appl. No.: 10/240,237

(22) PCT Filed: Mar. 29, 2001
(86) PCT No.: PCT/GB01/01435

(30) Foreign Application Priority Data
Mar. 30, 2000 (GB) 0007588.7

(51) Int. Cl. A61K 31/5377; A61K 31/496; A61K 31/365; C07D 413/02; C 07D 4 5/02

(52) U.S. Cl. 514/231.5; 514/471; 514/254.1; 544/152; 544/374; 549/321

(57) ABSTRACT

New N-acyl homoserine lactone compounds of the formula (I) in whichR is an acyl group of the formula (II) wherein one of R¹ and R² is H and the other is selected from OR⁴, SR⁴ and NHR⁴, wherein R⁴ is H or 1-6C alkyl, or R¹ and R² together with the carbon atom to which they are joined form a keto group, and R³ containing from 8 to 11 carbon atoms and is optionally substituted by one or more substituent groups selected from halo, 1-6C alkoxy, carboxy, 1-6C alkoxycarbonyl, carbamoyl optionally mono- or disubstituted at the N atom by 1-6C alkyl and NR⁵R⁶ wherein each of R⁵ and R⁶ is selected from H and 1-6C alkyl or R⁵ and R⁶ together with the N atom form a morpholino or piperazino group, or and enantiomer thereof, with the proviso that R is not a 3-oxododecanoyl group, having immuosuppressant properties are disclosed. The compounds are shown to have an inhibitory effect on lymphocyte proliferation and down-regulate TNF-α secretion by monocytes/macrophages in the animal body, including the human body. Pharmaceutical composition comprising N-acyl homoserine lactones are also described.

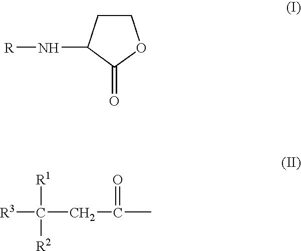

The invention relates to N-acyl homoserine lactones which have immunosuppressant properties and to pharmaceutical compositions containing them.

Immunosuppressant compounds induce an inhibition of the immune response system. Compounds which are known to exhibit immunosuppressant activity include the fungal metabolite Cyclosporin A and the macrolide antibiotic (a metabolite from Streptomyces tsukabaensis) termed FK506. Both of these agents have been used clinically and experimentally to suppress the immune system in transplantation and in the treatment of a number of diseases.

Autoimmune diseases are disorders where the host discrimination of “self” versus “non-self” breaks down and the individual's immune system (both acquired and innate components) attacks self tissues. These diseases range from common entities such as rheumatoid arthritis, thyroid autoimmune disease and type 1 diabetes mellitus to less common entities such as multiple sclerosis and to rarer disorders such as myasthenia gravis. Advances in basic biomedical science and, in particular, in immunology have indicated that the main and fundamental lesion responsible for the induction and persistence of most autoimmune diseases resides within auto-reactive proliferating T lymphocytes. In fact, the majority of autoimmune diseases are linked to a loss of T cell homeostasis. The healthy immune system is held in balanced equilibrium, apparently by the contra-suppressive production of cytokines by T helper 1 (Th1) and T helper 2 (Th2) lymphocyte subsets. In autoimmunity, Th1 cytokines predominate; in allergy, Th2 cytokines take their place. A cytokine intimately associated with the development of Th1 biased responses and, consequently, autoimmune disease is TNF-a.

Both Cyclosporin A and FK506 have been used clinically in the treatment of autoimmune diseases with encouraging results.

The currently available immunosuppressant drugs have the disadvantage of a narrow therapeutic index, i.e., toxicity versus clinical benefit. The compounds are known to bepnephrotoxic, neurotoxic and potentially diabetogenic and this has limited their use in the fields mentioned above. Problems also exist with the administration of these compounds, their bioavailability and the monitoring of their levels both clinically and in the laboratory.

We disclosed, in WO-A-95/01175, a class of compounds which exhibit antiallergic activity and inhibit the release of histamine, having the generic formula

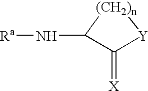

where: n is 2 or 3; Y is O, S or NH; X is O, S or NH; and R^{a} is C₁-C₁₈ alkyl or acyl which may be substituted.

Some of these compounds, and methods for their preparation, were previously disclosed in WO-A-92/18614 although that document discloses only that the compounds act as autoinducers and as agents for the control of gene expression. Compounds in the same series are also mentioned in Journal of Bacteriology, volume 175, number 12, June 1993, pages 3856 to 3862 but again there is no teaching that they might have any effect outside micro-organisms.

G. Papaccio, Diabetes Res. Clin. Pract. vol.13, no.1, 1991, pages 95-102 discloses the use of N-acetylhomocysteine thiolactone as an enhancer of superoxide dismutase in an attempt to increase protection against chemically induced diabetes.

The use of N-acetylhomocysteine thiolactone to modify the IgE molecule is taught by J. Ljaljevic et al in Od. Med. Nauka, vol.24, 1971, pages 137-143 and Chemical Abstracts, vol.78, no.7, February 1973, abstract no. 41213a. However, there is no suggestion in this paper of immunosuppression or of the inhibition of histamine release.

U.S. Pat. No. 5,591,872 discloses the compound N-(3-oxododecanoyl) homoserine lactone as an autoinducer molecule. In “Infection and Immunity”, vol.66, no.1, January 1998, the authors report the action of N-(3-oxododecanoyl)homoserine lactone (OdDHL) in inhibiting the concavalin A mitogen stimulated proliferation of murine spleen cells and TNF-a production by LPS-stimulated adherent murine peritoneal macrophages.

We have now discovered a subclass of N-acyl homoserine lactones that exhibits an immunosuppressant activity greater than that exhibited by similar compounds outside of this subclass.

According to one aspect, the present invention provides a compound of the formula I

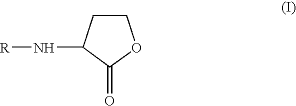

in which R is an acyl group of the formula II

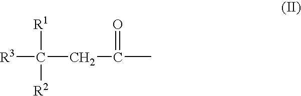

wherein one of R¹ and R² is H and the other is selected from OR⁴, SR⁴ and NHR⁴, wherein R⁴ is H or 1-6C alkyl, or R¹ and R² together with the carbon atom to which they are joined form a keto group, and R³ is a straight or branched chain, saturated or unsaturated aliphatic hydrocarbyl group containing from 8 to 11 carbon atoms and is optionally substituted by one or more substituent groups selected from halo, 1-6C alkoxy, carboxy, 1-6C alkoxycarbonyl, carbamoyl optionally mono- or disubstituted at the N atom by 1-6C alkyl and NR⁵R⁶ wherein each of R⁵ and R⁶ is selected from H and 1-6C alkyl or R⁵ and R⁶ together with the N atom form a morpholino or piperazino group, or any enantiomer thereof, with the proviso that R is not a 3-oxododecanoyl group.

The compounds of the present invention are capable of modulating the immune response in the living animal body, including human. In particular, they have an inhibitory effect on lymphocyte proliferation in humans and down-regulate TNF-α secretion by monocytes/macrophages and, in consequence, the activation of Th1 lymphocytes in humans. The present invention, therefore, provides a pharmaceutical composition comprising a therapeutically-effective amount of a compound of the invention as described herein, including an enantiomer thereof, together with a pharmaceutically-acceptable carrier or diluent.

A further aspect of the invention provides the use of a compound of the invention, including an enantiomer thereof, for the manufacture of a medicament for the treatment of a disease of a living animal body including human which disease is responsive to the activity of an immunosuppressant, for example an autoimmune disease. A yet further aspect of the invention relates to a method of treating a disease of a living animal body, including a human, which disease is responsive to the activity of an immunosuppressant, e.g., an autoimmune disease, which method comprises administering to the living animal body, including human, a therapeutically-effective amount of a compound according to the invention, as described herein including an enantiomer thereof. Compounds of the invention have the general formula I given above. The group R in the formula I has the formula II

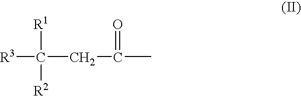

In formula II according to a first preferred embodiment one of R¹ and R² is H and the other is selected from OR⁴, SR⁴ and NHR⁴, in which R⁴ is H or a 1-6C alkyl group. Preferably, R⁴ is H. Such a definition of R¹ and R² gives rise to chirality at the carbon atom to which R¹ and R² are attached (C-3). The compounds of the invention can, thus, be in the form of racemates, optically active isomers or mixtures thereof. According to a particular preferred embodiment one of R¹ and R² is H and the other is OH.

According to this first preferred embodiment the group R³ in formula II is a straight or branched chain 8 to 11 C aliphatic hydrocarbyl group which is saturated or which may be ethylenically unsaturated. The group may, further, be substituted by one or more substituent groups selected from halo, for example F, Cl, Br or I; 1-6C alkoxy, for example methoxy, ethoxy, n-propoxy, iso-propoxy, n-butoxy, iso-butoxy and tert-butoxy; carboxy including salts thereof, 1-6C alkoxycarbonyl, for example methoxycarbonyl, carbamoyl, for example N,N-dimethylcarbamoyl and NR⁵R⁶, wherein R⁵ and R⁶ are each selected from H and 1-6C alkyl or R⁵ and R⁶ together with the nitrogen atom to which they are attached form a morpholino group or a piperazino ring, optionally substituted at the 4-N by a methyl group. A particularly preferred R³ group in formula 11 above is a straight chain or branched chain 8 to 11C alkyl group which is optionally substituted by one substituent selected from Br, carboxy including salts thereof, and methoxycarbonyl. The substituent is typically, though not necessarily, attached in a terminal position on the alkyl group. Alternatively, the R³ group is a straight chain or branched chain 8-11 C alkenyl group, preferably monoethenically unsaturated, which may be substituted by a substituent selected from Br, carboxy including a salt thereof, and methoxycarbonyl. Again, the substituent is typically, though not necessarily, attached in a terminal position on the alkenyl group.

In formula II above according to a second preferred embodiment the groups R¹ and R² together form an oxo group (═O) such that a keto group exists at the C-3 position in the acyl group. In such a case the group R³ in formula II will typically be:

(a) an optionally-substituted, saturated or ethylenically-unsaturated, straight or branched chain 8C aliphatic hydrocarbyl group;

(b) a substituted, saturated, straight or branched chain 9C aliphatic hydrocarbyl group;

(c) an optionally-substituted, ethylenically-unsaturated, straight or branched chain 9C aliphatic hydrocarbyl group;

(d) an optionally-substituted, saturated or ethylenically-unsaturated, straight or branched chain 10C aliphatic hydrocarbyl group; or

(e) an optionally-substituted, saturated or ethylenically-unsaturated, straight or branched chain 11C aliphatic hydrocarbyl group.

In the case where the group R³ is substituted, it will be substituted by one or more substituent groups selected from halo, for example F, Cl, Br or I; 1-6C alkoxy, for example methoxy, ethoxy, n-propoxy, iso-propoxy, n-butoxy, iso-butoxy and tert-butoxy; carboxy including salts thereof, 1-6C alkoxycarbonyl, for example methoxycarbonyl, carbamoyl, for example N,N-dimethylcarbamoyl, and NR⁵R⁶, wherein R⁵ and R⁶ are each selected from H and 1-6C alkyl or R⁵ and R⁶ together with the nitrogen atom to which they are attached form a morpholino group or a piperazino ring, optionally substituted at the 4-N by a methyl group.

According to one preferred embodiment the R³ group in formula 11 above is a straight chain or branched chain 8, 10 or 11C alkyl group which is optionally substituted by one substituent selected from Br, carboxy including salts thereof, and methoxycarbonyl. According to another preferred embodiment the R³ in formula II above is a straight chain or branched chain 9C alkyl group which is substituted by one substituent selected from Br, carboxy including salts thereof and methoxycarbonyl. The substituent is typically, though not necessarily, attached in a terminal position on the alkyl group.

According to yet another preferred embodiment the R³ group is a straight chain or branched chain 8-11C alkenyl group, preferably monoethenically unsaturated, which may be substituted by a substituent selected from Br, carboxy including a salt thereof, and methoxycarbonyl. The substituent is typically, though not necessarily, attached in a terminal position on the alkenyl group.

Examples of acyl groups R of formula II above in which R³ is a saturated hydrocarbyl group include:—

3-oxoundecanoyl;

11-bromo-3-oxoundecanoyl;

10-methyl-3-oxoundecanoyl;

6-methyl-3-oxoundecanoyl;

3-hydroxydodecanoyl;

12-bromo-3-oxododecanoyl;

3-oxotridecanoyl;

13-bromo-3-oxododecanoyl;

3-hydroxytetradecanoyl;

3-oxotetradecanoyl;

14-bromo-3-oxotetradecanoyl; and

13-methoxycarbonyl-3-oxotridecanoyl.

Examples of acyl groups R of formula II above in which R³ is an ethylenically unsaturated hydrocarbyl group include:—

3-oxo-12-tridecenoyl;

3-oxo-7-tetradecenoyl;

3-hydroxy-7-tetradecenoyl;

3-oxo-9-tetradecenoyl;

3-hydroxy-9-tetradecenoyl;

3-oxo-10-tetradecenoyl;

3-hydroxy-10-tetradecenoyl;

3-oxo-11-tetradecenoyl;

3-hydroxy-11-tetradecenoyl;

3-oxo-13-tetradecenoyl; and

3-hydroxy-13-tetradecenoyl.

The compounds of the present invention having the 3-oxo group may, in general, be prepared by a method comprising the steps of:

(1) reacting an acid having the general formula R³COOH, where R³ is as defined above, with Meldrum's acid (2,2-dimethyl-1,3-dioxane4,6-dione) in the presence of 4-dimethylaminopyridine and N,N¹-dicyclohexylcarboxdiimide in a dry organic solvent, such as dry dichloromethane, to give the acylated Meldrum's acid; and

(2) reacting the acylated Meldrum's acid with L-homoserine lactone hydrochloride in an organic solvent, e.g., acetonitrile, to give the N-(3-oxoacylated)-L-homoserine lactone.

Where the appropriate acid is not available it may be prepared by, for instance, oxidising the appropriate alcohol using chromic acid.

The corresponding N-(3-hydroxyacylated)-L-homoserine lactone may be prepared by reducing the N-(3-oxoacylated)-L-homoserine lactone using sodium cyanoborohydride in acid conditions.

As mentioned above, the compounds of the present invention have use as pharmaceutically active ingredients in the treatment of an animal body, including the human body, suffering from a disease or disorder which is responsive to the activity of an immunosuppressant, particularly for the treatment of an autoimmune disease such as psoriasis, multiple sclerosis and rheumatoid arthritis. The dosage administered to the animal body in need of therapy will, of course, depend on the actual active compound used, the mode of treatment and the type of treatment desired as well as on the body mass. The active compound may, of course, be administered on its own or in the form of an appropriate medicinal composition containing, for instance, an appropriate pharmaceutical carrier or diluent. Other substances can, of course, also be employed in such medicinal compositions, such as antioxidants and stabilisers, the use of which is well known to persons skilled in the art.

The present invention, as mentioned above also encompasses the use of a compound of the invention or the known compound N-(3-oxododecanoyl)-homoserine lactone in a treatment of psoriasis. Such an active compound will, typically, be formulated for topical application to the patient, for instance in the form of ointment, cream or lotion.

In view of their apparently selective immune suppressant properties the compounds described herein, can also be used in a vaccine preparation as an adjuvant, in situations where enhanced Th2 responses would be beneficial, for example when vaccinating against worm infection in humans and domestic animals.

### EXAMPLES

### Example 1

### N-(3-oxoundecanoyl)-L-homoserine lactone (OuDHL)

To a solution of nonanoic acid (2 mmol) in dry dichloromethane (20 ml) was added 4-dimethylaminopyridine (2.1 mmol), N,N′-dicyclohexylcarbodiimide (2.2 mmol) and Meldrum's acid (2 mmol). The solution was stirred at room temperature overnight and then filtered to remove the precipitated dicyclohexylurea. The filtrate was evaporated to dryness and the residue redissolved in ethyl acetate. The ethyl acetate solution was washed with 2M hydrochloric acid, dried over anhydrous magnesium sulphate and concentrated to afford the nonanoyl Meldrum's acid.

To a stirred solution of the nonanoyl Meldrum's acid (1 mmol) in acetonitrile (30 ml) was added L-homoserine lactone hydrochloride (1 mmol) and triethylamine (1.2 mmol). The mixture was stirred for 2 h and then refluxed for a further 3 h. The solvent was removed by rotary evaporation to give a residue that was redissolved in ethyl acetate. The organic solution was sequentially washed with saturated sodium hydrogen carbonate solution, 1 M potassium hydrogen sulphate solution and saturated sodium chloride solution. After drying over anhydrous magnesium sulphate, the organic extract was evaporated to dryness and the residue purified by preparative layer chromatography on silica plates.

Spectral Data

N-(3-Oxoundecanovl)-L-homoserine Lactone

¹H NMR (250 MHz, CDCl₃) δ 0.9 (3H, t, CH₃), 1.27 (10H, m, CH₃(CH₂)₅), 1.59 (2H, m, CH₂CH₂CO), 2.22 (1H, m, 4α-H), 2.52 (2H, t, CH₂CO), 2.76 (1H, m, 4β-H), 3.47 (2H, s, COCH₂CO), 4.27 (1H, m, 5α-H), 4.48 (1H, td, 5β-H), 4.58 (1H, m, 3-H), 7.64 (1H, d, NH).

The procedure described above in Example 1 was followed to prepare other N-(3-oxoacylated)-L-homoserine lactones as described below using, in each case, the appropriate carboxylic acid.

### Example 2

### N-(11-Bromo-3-oxoundecanoyl)-L-homoserine Lactone (11Br OuDHL)

ES-MS m/z 362.2 & 364.2 (MH+, C₁₅H₂₅NO₄Br requires m/z 362 & 364); ¹H NMR (250 MHz, CDCl₃) δ 1.27 (8H, m, BrCH₂CH₂(CH₂)₄), 1.45 (2H, m, BrCH₂CH₂CH₂) 1.59 (2H, m, CH₂CH₂CO), 2.22 (1H, m, 4α-H), 2.52 (2H, t, CH₂CO), 2.76 (1H, m, 4β-H), 3.47 (2H, s, COCH₂CO), 3.53 (2H, t, BrCH₂) 4.27 (1H, m, 5α-H), 4.48 (1H, td, 5β-H), 4.58 (1H, m, 3-H), 7.64 (1H, d, NH).

### Example 3

### N-(10-Methyl-3-oxoundecanoyl)-L-homoserine lactone (10MeOuDHL)

ES-MS m/z 298.0 (MH+,C₁₆H₂₈NO₄ requires m/z 298.0); ¹H NMR (90 MHz, CDCl₃) δ0.8 (6H, d, (CH₃)₂CH), 1.2 (9H, m, CH(CH₂)₄), 1.7 (2H, m, CH₂CH₂CO), 2.2 (1H, m, 4α-H), 2.45 (2H, t, CH₂CO), 2.65 (1H, m, 4β-H), 3.4 (2H, s, COCH₂CO), 4.0-4.8 (3H, m, 5α-H, 5β-H, 3-H), 7.65 (1H, d, NH).

### Example 4

### N-(10-Methoxycarbonyl-3-oxodecanoyl)-L-homoserine lactone (10(MeO₂C)ODHL)

ES-MS m/z 328.3 (MH+, C₁₆H₂₆NO₆ requires m/z 328); ¹H NMR (250 MHz, CDCl₃) δ 1.27 (6H, m, CH₃OCOCH₂CH₂(CH₂)₃), 1.59 (4H, m, CH₂CH₂CO & CH₃OCOCH₂CH₂), 2.22 (1H, m, 4α-H), 2.25 (2H, t, CH₃OCOCH₂), 2.52 (2H, t, CH₂CO), 2.76 (1H, m, 4β-H), 3.47 (2H, s, COCH₂CO), 3.58 (3H, s, CH₃O), 4.27 (1H, m, 5α-H), 4.48 (1H, td, 5β-H), 4.58 (1H, m, 3-H), 7.64 (1H, d, NH).

### Example 5

### N-(6-Methyl-3-oxoundecanoyl)-L-homoserine lactone (6MeOuDHL)

ES-MS m/z 298.2 (MH+, C₁₆H₂₈NO₄ requires m/z 298); ¹H NMR (250 MHz, CDCl₃) δ 0.9 (6H, t&d, CH₃CH₂&CHCH₃), 1.27 (10H, m, CH₃(CH₂)₅), 1.59 (2H, m, CH₂CH₂CO), 2.22 (1H, m, 4α-H), 2.52 (2H, t, CH₂CO), 2.76 (1H, m, 4β-H), 3.47 (2H, s, COCH₂CO), 4.27 (1H, m, 5α-H), 4.48 (1H, td, 5β-H), 4.58 (1H, m, 3-H), 7.64 (1H, d, NH).

### Example 6

### N-(3-Oxododecanoyl)-L-homoserine lactone (OdDHL)

¹H NMR (250 MHz, CDCl₃) δ 0.9 (3H, t, CH₃), 1.27 (12H, m, CH₃(CH₂)₆), 1.59 (2H, m, CH₂CH₂CO), 2.22 (1H, m, 4α-H), 2.52 (2H, t, CH₂CO), 2.76 (1H, m, 4β-H), 3.47 (2H, s, COCH₂CO), 4.27 (1H, m, 5α-H), 4.48 (1H, td, 5β-H), 4.58 (1H, m, 3-H), 7.64 (1H, d, NH).

### Example 7

### N-(12-Bromo-3-oxododecanoyl)-L-homoserine Lactone (12BrOdDHL)

¹H NMR (250 MHz, CDCl₃) δ 1.27 (10H, m, BrCH₂CH₂(CH₂)₅), 1.45 (2H, m, BrCH₂CH₂CH₂), 1.59 (2H, m, CH₂CH₂CO), 2.22 (1H, m, 4α-H), 2.52 (2H, t, CH₂CO), 2.76 (1H, m, 4β-H), 3.47 (2H, s, COCH₂CO), 3.53 (2H, t, BrCH₂), 4.27 (1H, m, 5α-H), 4.48 (1H, td, 5β-H), 4.58 (1H, m, 3-H), 7.64 (1H, d, NH).

### Example 8

### N-(3-Oxotridecanoyl)-L-homoserine Lactone (OtriDHL)

¹H NMR (250 MHz, CDCl₃) δ 0.9 (3H, t, CH₃), 1.27 (14H, m, CH₃(CH₂)₇), 1.59 (2H, m, CH₂CH₂CO), 2.22 (1H, m, 4α-H), 2.52 (2H, t, CH₂CO), 2.76 (1H, m, 4β-H), 3.47 (2H, s, COCH₂CO), 4.27 (1H, m, 5α-H), 4.48 (1H, td, 5β-H), 4.58 (1H, m, 3-H), 7.64 (1H, d, NH).

### Example 9

### N-(13-Bromo-3-oxotridecanoyl)-L-homoserine Lactone (13Br-OtriDHL)

ES-MS m/z 390.6 & 392.6 (MH+,C₁₇H₂₉NO₄Br requires m/z 390 & 392); ¹H NMR (250 MHz, CDCl₃) δ 1.27 (12H, m, BrCH₂CH₂(CH₂)₆), 1.45 (2H, m, BrCH₂CH₂CH₂) 1.59 (2H, m, CH₂CH₂CO), 2.22 (1H. m, 4α-H), 2.52 (2H, t, CH₂CO), 2.76 (1H, m, 4β-H), 3.47 (2H, s, COCH₂CO), 3.53 (2H, t, BrCH₂) 4.27 (1H, m, 5α-H), 4.48 (1H, td, 5β-H), 4.58 (1H, m, 3-H), 7.64 (1H, d, NH).

### Example 10

### N-(3-Oxo-12-tridecenoyl)-L-homoserine lactone (12 dB-OtriDHL)

¹H NMR (250 MHz, CDCl₃) δ 1.27 (10H, m, CH₂CH(CH₂)₅), 1.59 (2H, m, CH₂CH₂CO), 2.00 (2H, m, CH₂CHCH₂) 2.22 (1H, m, 4α-H), 2.52 (2H, t, CH₂CO), 2.76 (1H, m, 4β-H), 3.47 (2H, s, COCH₂CO), 4.27 (1H, m, 5α-H), 4.48 (1H, td, 5β-H), 4.58 (1H, m, 3-H), 4.93 (2H, m, CH₂CH), 5.86 (1H, m, CH₂CH), 7.64 (1H, d, NH).

### Example 11

### N-(3-Oxotetradecanoyl)-L-homoserine Lactone (OtDHL)

¹H NMR (250 MHz, CDCl₃) δ 0.9 (3H, t, CH₃), 1.27 (16H, m, CH₃(CH₂)₈), 1.59 (2H, m, CH₂CH₂CO), 2.22 (1H, m, 4α-H), 2.52 (2H, t, CH₂CO), 2.76 (1H, m, 4β-H), 3.47 (2H, s, COCH₂CO), 4.27 (1H, m, 5α-H), 4.48 (1H, td, 5β-H), 4.58 (1H, m, 3-H), 7.64 (1H, d, NH).

### Example 12

### N-(14-Bromo-3-oxotetradecanoyl)-L-homoserine Lactone (14BrOtDHL)

¹H NMR (250 MHz, CDCl₃) δ 1.27 (14H, m, BrCH₂CH₂(CH₂)₇), 1.45 (2H, m, BrCH₂CH₂CH₂), 1.59 (2H, m, CH₂CH₂CO), 2.22 (1H, m, 4α-H), 2.52 (2H, t, CH₂CO), 2.76 (1H, m, 4β-H), 3.47 (2H, s, COCH₂CO), 3.53 (2H, t, BrCH₂), 4.27 (1H, m, 5α-H), 4.48 (1H, td, 5β-H), 4.58 (1H, m, 3-H), 7.64 (1H, d, NH).

### Example 13

### N-(13-Methoxycarbonyl-3-oxotridecanoyl)-L-homoserine Lactone ((13MeO₂C)OtriDHL)

ES-MS m/z 370.3 (MH+, C₁₉H₃₂NO₆ requires m/z 370); ¹H NMR (250 MHz, CDCl₃) δ 1.27 (10H, m, CH₃OCOCH₂CH₂(CH₂)5), 1.59 (4H, m, CH₂CH₂CO & CH₃OCOCH₂CH₂), 2.22 (¹H, m, 4α-H), 2.25 (2H, t, CH₃OCOCH₂), 2.52 (2H, t, CH₂CO), 2.76 (1H, m, 4β-H), 3.47 (2H, s, COCH₂CO), 3.58 (3H, s, CH₃O), 4.27 (1H, m, 5α-H), 4.48 (1H, td, 5β-H), 4.58 (1H, m, 3-H), 7.64 (1H, d, NH).

### Example 14

### N-(3-Oxo-7-tetradecenoyl)-L-homoserine Lactone (7cisOtDHL)

¹H NMR (250 MHz, CDCl₃) δ 0.9 (3H, t, CH₃), 1.27 (8H, m, CH₃(CH₂)₄), 1.59 (2H, m, CH₂CH₂CO), 2.22 (1H, m, 4α-H), 1.95 (4H, m, CH₂CHCHCH₂), 2.52 (2H, t, CH₂CO), 2.76 (1H, m, 4β-H), 3.47 (2H, s, COCH₂CO), 4.27 (1H, m, 5α-H), 4.48 (1H, td, 5β-H), 4.58 (1H, m, 3-H), 5.32 (2H, m, CHCH), 7.64 (1H, d, NH).

### Example 15

### N-(3-Oxo-9-tetradecenoyl)-L-homoserine Lactone (9cisOtDHL)

ES-MS m/z 324.7 (MH+, C₁₈H₃₀NO₄ requires m/z 324); ¹H NMR (250 MHz, CDCl₃) δ 0.9 (3H, t, CH₃), 1.27 (8H, m, CH₃(CH₂)₂ & (CH₂)₂CH₂CO), 1.59 (2H, m, CH₂CH₂CO), 2.22 (1H, m, 4α-H), 1.95 (4H, m, CH₂CHCHCH₂), 2.52 (2H, t, CH₂CO), 2.76 (1H, m, 4β-H), 3.47 (2H, s, COCH₂CO), 4.27 (1H, m, 5α-H), 4.48 (1H, td, 5β-H), 4.58 (1H, m, 3-H), 5.32 (2H, m, CHCH), 7.64 (1H, d, NH).

### Example 16

### N-(3-Oxo-10-tetradecenoyl)-L-homoserine lactone (10cisOtDHL)

ES-MS m/z 323.8 (MH+, C₁₈H₃₀NO₄ requires m/z 324); ¹H NMR (250 MHz, CDCl₃) δ 0.9 (3H, t, CH₃), 1.27 (8H, m, CH₃CH₂ & (CH₂)₃CH₂CO), 1.59 (2H, m, CH₂CH₂CO), 2.22 (1H, m, 4α-H), 1.95 (4H, m, CH₂CHCHCH₂), 2.52 (2H, t, CH₂CO), 2.76 (1H. m, 4β-H), 3.47 (2H, s, COCH₂CO), 4.27 (1H, m, 5α-H), 4.48 (1H, td, 5β-H), 4.58 (1H, m, 3-H), 5.32 (2H, m, CHCH, 7.64 (1H, d, NH).

### Example 17

### N-(3-Oxo-11-tetradecenoyl)-L-homoserine Lactone (11cisOtDHL)

ES-MS m/z 324.3 (MH+, C₁₈H₃₀NO₄ requires m/z 324); ¹H NMR (250 MHz, CDCl₃) δ 0.9 (3H, t, CH₃), 1.27 (8H, m, (CH₂)₄CH₂CO), 1.59 (2H, m, CH₂CH₂CO), 2.22 (1H, m, 4α-H), 1.95 (4H, m, CH₂CHCHCH₂), 2.52 (2H, t, CH₂CO), 2.76 (1H, m, 4β-H), 3.47 (2H, s, COCH₂CO), 4.27 (1H, m, 5α-H), 4.48 (1H, td, 5β-H), 4.58 (1H, m, 3-H), 5.32 (2H, m, CHCH), 7.64 (1H, d, NH).

### Example 18

### N-(3-Oxo-13-tetradecenoyl)-L-lactone Homoserine (13 dbOtDHL)

ES-MS m/z 323.6 (MH+, C₁₈H₃₀NO₄ requires m/z 324); ¹H NMR (250 MHz, CDCl₃) δ 1.27 (12H, m, CH₂CH(CH₂)₆), 1.59 (2H, m, CH₂CH₂CO), 2.00 (2H, m, CH₂CHCH₂), 2.22 (1H, m, 4α-H), 2.52 (2H, t, CH₂CO), 2.76 (1H, m, 4β-H), 3.47 (2H, s, COCH₂CO), 4.27 (1H, m, 5α-H), 4.48 (1H, td, 5β-H), 4.58 (1H, m, 3-H), 4.93 (2H, m, CH₂CH), 5.86 (1H, m, CH₂CH), 7.64 (1H, d, NH).

### Example 19

### N-(12-Hydroxy-3-oxododecanoyl)-L-homoserine Lactone (12OHOdDHL).

Using 10-acetoxydecanoic acid in the general procedure as described above in Example 1 afforded the N-(12-acetoxy-3-oxododecanoyl)-L-homoserine lactone. The latter when refluxed in 1M hydrochloric acid, yielded the title product.

¹H NMR (250 MHz, CDCl₃) δ 1.27 (12H, m, HOCH₂(CH₂)₆), 1.59 (2H, m, CH₂CH₂CO), 1.89 (1H, t, OH), 2.22 (1H, m, 4α-H), 2.52 (2H, t, CH₂CO), 2.76 (1H, m, 4β-H), 3.47 (2H, s, COCH₂CO), 3.60 (2H, t, HOCH₂), 4.27 (1H, m, 5α-H), 4.48 (1H, td, 5β-H), 4.58 (1H, m, 3-H), 7.64 (1H, d, NH).

### Example 20

### N-[11-(N, N-Dimethylcarbamoyl)-3-oxoundecanoyl]-L-homoserine Lactone (11Me₂NCO)OuDHL)

9-(N,N-Dimethylcarbamoyl)nonanoic acid was prepared as follows:

To a solution of the decandioic acid mono methyl ester (2 mmol) in dry tetrahydrofuran (20 ml) was added 1-hydroxybenzotriazole (1 mmol), N,N′-dicyclohexylcarbodiimide (1.1 mmol) and N,N-dimethylamine (2M solution in tetrahydrofuran, 2 ml). The mixture was stirred overnight and then filtered. The solvent was removed in vacuo and the residue redissolved in ethyl acetate. The ethyl acetate solution was washed sequentially with saturated sodium hydrogen carbonate solution, 1 M potassium hydrogen sulphate solution and saturated sodium chloride solution. After drying over anhydrous magnesium sulphate and removal of the solvent, the product was purified by preparative layer chromatography on silica plates in ethyl acetate to afford methyl 9-(N,N-dimethylcarbamoyl)nonanoate.

The methyl ester was stirred in 1M sodium hydoxide (20 ml) and methanol (10 ml) solution overnight. The methanol was removed in vacuo and the solution acidified to pH 1 with 2M hydrochloric acid. The product was extracted with dichloromethane (3×10 ml) and the combined extracts were dried over anhydrous magnesium sulphate and concentrated by rotary evaporation to give the desired 9-(N,N-dimethylcarbamoyl)nonanoic acid.

¹H NMR (250 MHz, CDCl₃) δ 1.27 (8H, m, NCOCH₂CH₂CH₂(CH₂)₄), 1.59 (4H, m, CH₂CH₂CO&NCOCH₂CH₂), 2.22 (1H, m, 4α-H), 2.40 (2H, t, NCOCH₂ 2.52 (2H, t, CH₂CO), 2.76 (1H, m, 4β-H), 3.47 (2H, s, COCH₂CO), 4.27 (1H, m, 5α-H), 4.48 (1H, td, 5β-H), 4.58 (1H, m, 3-H), 7.64 (1H, d, NH).

### Example 21

### N-(3-Hydroxydodecanoyl)-L-homoserine Lactone (HdDHL)

N-(3-Oxododecanoyl)-L-homoserine lactone (1 mmol) was dissolved in methanol (10 ml) and the solution made acidic (pH 3-4) with 2 M HCl-methanol. Sodium cyanoborohydride (2.5 mmol) was added in one lot with stirring and the reaction mixture maintained at pH 3˜4 by the occasional addition of 2 M HCl-methanol. After 2 hours, solvent was removed in vacuo and ethyl acetate extracts (3×10 ml) of the residue were combined, dried (MgSO₄) and evaporated to yield the title hydroxy derivatives. The products were purified by preparative layer chromatography on silica plates in CHCl₃—MeOH (9:1).

¹H NMR (250 MHz, CDCl₃) δ 0.87 (3H, t, CH₃), 1.27 (14H, m, (CH₂)₇CH₂CHOH), 1.56 (2H, m, CH₂CHOH), 2.22 (1H, m, 4α-H), 2.38 (2H, m, CHOHCH₂CO), 2.76 (1H, m, 4β-H), 3.15 (1H, m, CHOH), 3.98 (1H, brs, OH), 4.27 (1H, m, 5α-H), 4.48 (1H, td, 5β-H), 4.58 (1H, m, 3-H), 7.64 (1H, d, NH).

Other N-(3-hydroxyacylated)-L-homoserine lactones were prepared according to the procedure described above in Example 21 by reducing the appropriate -3-oxo compound as follows.

### Example 22

### N-(3-Hvdroxytetradecanoyl)-L-homoserine Lactone (HtDHL)

ES-MS m/z 328.1 (MH+, C₁₈H₃₄NO₄ requires m/z 328); ¹H NMR (250 MHz, CDCl₃) δ 0.87 (3H, t, CH₃), 1.27 (18H, m, (CH₂)₉CH₂CHOH), 1.56 (2H, m, CH₂CHOH), 2.22 (1H, m, 4α-H), 2.38 (2H, m, CHOHCH₂CO), 2.76 (1H, m, 4β-H), 3.15 (1H, m, CHOH), 3.98 (1H, brs, OH), 4.27 (1H, m, 5α-H), 4.48 (1H, td, 5β-H), 4.58 (1H, m, 3-H), 7.64 (1H, d, NH).

### Example 23

### N-(3-Hydroxy-7-tetradecenoyl)-L-homoserine Lactone (7cisHtDHL)

¹H NMR (250 MHz, CDCl₃) δ 0.9 (3H, t, CH₃), 1.27 (10H, m, CH₃(CH₂)₄ & CH₂CH₂CHOH), 1.40 (2H, m, CH₂CHOH), 2.00 (4H, m, CH₂CHCHCH₂), 2.22 (1H, m, 4α-H), 2.38 (2H, m, CHOHCH₂CO), 2.76 (1H, m, 4β-H), 3.15 (1H, m, CHOH), 3.98 (1H, brs, OH), 4.27 (1H, m, 5α-H), 4.48 (1H, td, 5β-H), 4.58 (1H, m, 3-H), 5.32 (2H, m, CHCH), 7.64 (1H, d, NH).

### Example 24

### N-(3-Hydroxy-9-tetradecenoyl)-L-homoserine Lactone (9cisHtDHL)

ES-MS m/z 326.3 (MH+, C₁₈H₃₂NO₄ requires m/z 326); ¹H NMR (250 MHz, CDCl₃) δ 0.9 (3H, t, CH₃), 1.27 (10H, m, CH₃(CH₂)₂ & (CH₂)₃CH₂CHOH), 1.40 (2H, m, CH₂CHOH), 2.00 (4H, m, CH₂CHCHCH₂), 2.22 (1H, m, 4α-H), 2.38 (2H, m, CHOHCH₂CO), 2.76 (1H, m, 4β-H), 3.15 (1H, m, CHOH), 3.98 (1H, brs, OH), 4.27 (1H, m, 5α-H), 4.48 (1H, td, 5β-H), 4.58 (1H, m, 3-H), 5.32 (2H, m, CHCH), 7.64 (1H, d, NH).

### Example 25

### N-(3-Hydroxy-10-tetradecenoyl)-L-homoserine Lactone (10cisHtDHL)

ES-MS m/z 325.6 (MH+, C₁₈H₃₂NO₄ requires m/z 326); ¹H NMR (250 MHz, CDCb₃) δ 0.9 (3H, t, CH₃), 1.27 (10H, m, CH₃CH₂ & (CH₂)₄CH₂CHOH), 1.40 (2H, m, CH₂CHOH), 2.00 (4H, m, CH₂CHCHCH₂), 2.22 (1H, m, 4α-H), 2.38 (2H, m, CHOHCH₂CO), 2.76 (1H, m, 4β-H), 3.15 (1H, m, CHOH), 3.98 (1H, brs, OH), 4.27 (1H, m, 5α-H), 4.48 (1H, td, 5β-H), 4.58 (1H, m, 3-H), 5.32 (2H, m, CHCH), 7.64 (1H, d, NH).

### Example 26

### N-(3-Hydroxy-11-tetradecenoyl)-L-homoserine Lactone (11cisHtDHL)

ES-MS m/z 325.7 (MH+, C₁₈H₃₂NO₄ requires m/z 326); ¹H NMR (250 MHz, CDCl₃) δ 0.9 (3H, t, CH₃), 1.27 (10H, m, (CH₂)₅CH₂CHOH), 1.40 (2H, m, CH₂CHOH), 2.00 (4H, m, CH₂CHCHCH₂), 2.22 (1H, m, 4α-H), 2.38 (2H, m, CHOHCH₂CO), 2.76 (1H, m, 4β-H), 3.15 (1H, m, CHOH), 3.98 (1H, brs, OH), 4.27 (1H, m, 5α-H), 4.48 (1H, td, 5β-H), 4.58 (1H, m, 3-H), 5.32 (2H, m, CHCH, 7.64 (1H, d, NH).

### Example 27

### N-(3-Hydroxy-13-tetradecenoyl)-L-homoserine Lactone (13 dbHtDHL)

ES-MS m/z 326.3 (MH+, C₁₈H₃₂NO₄ requires m/z 326); ¹H NMR (250 MHz, CDCl₃) δ1.27 (14H, m, (CH₂)₇CH₂CHOH), 1.40 (2H, m, CH₂CHOH), 2.00 (2H, m, CH₂CHCH₂), 2.22 (1H, m, 4α-H), 2.38 (2H, m, CHOHCH₂CO), 2.76 (1H, m, 4β-H), 3.15 (1H, m, CHOH), 3.98 (1H, brs, OH), 4.27 (1H, m, 5α-H), 4.48 (1H, td, 5β-H), 4.58 (1H, m, 3-H), 5.32 (2H, m, CHHCH, 5.83 (1H, m, CHHCH), 7.64 (1H, d, NH).

Immunomodulatory Activity of Homoserine Lactone Compounds

Materials and Methods

I. ConA Mitogen-Stimulated Proliferation of Murine Splenocytes

The concanavalin A (ConA) cell proliferation assay was used to assess the effect of homoserine lactone (HSL) compounds on T-cell activation and proliferation. Proliferation was assessed by the incorporation of [³H]-thymidine into DNA. Eight-week-old female BALB/c mice were obtained from Harlan (Bicester, Oxon, UK) and given food and water ad libitum. Splenocyte suspensions were prepared by removing the spleens and placing them into RPMI 1640 medium. The spleens were forced through 70-μm-pore-size wire gauzes using the plunger from a 5-ml syringe to produce a single cell suspension. The cells were pelleted by centrifugation, and erythrocytes were lysed with 0.017M Tris, 0.144M ammonium chloride buffer, pH 7.2. Leucocytes were washed twice with RPMI 1640 medium with 2% (vol/vol) foetal calf serum (FCS) and resuspended in complete cell culture medium (CTCM) consisting of RPMI 1640 medium with 5% FCS, 2 mM L-glutamine, and 5×10⁻⁵ M 2-mercaptoethanol. HSL compounds were tested at doubling down dilutions ranging from 1 mM to 0.1 μM in a final volume of 200 μl of CTCM, containing ConA (Sigma, Poole, UK) at 1 μg/ml and 100,000 spleen cells. Following incubation for 48 h at 37° C. in 5% CO₂-air, 0.25 μCi [³H]-thymidine (Amersham) in 10 μl volume made up in RPMI 1640 medium was added and the cells were incubated for a further 24 h. Cells were harvested onto fibreglass filters with a Packard filtermate harvester. After the addition of 25 μl of MicroScint-O (Packard) to each well the filters were counted with the Packard TopCount scintillation counter.

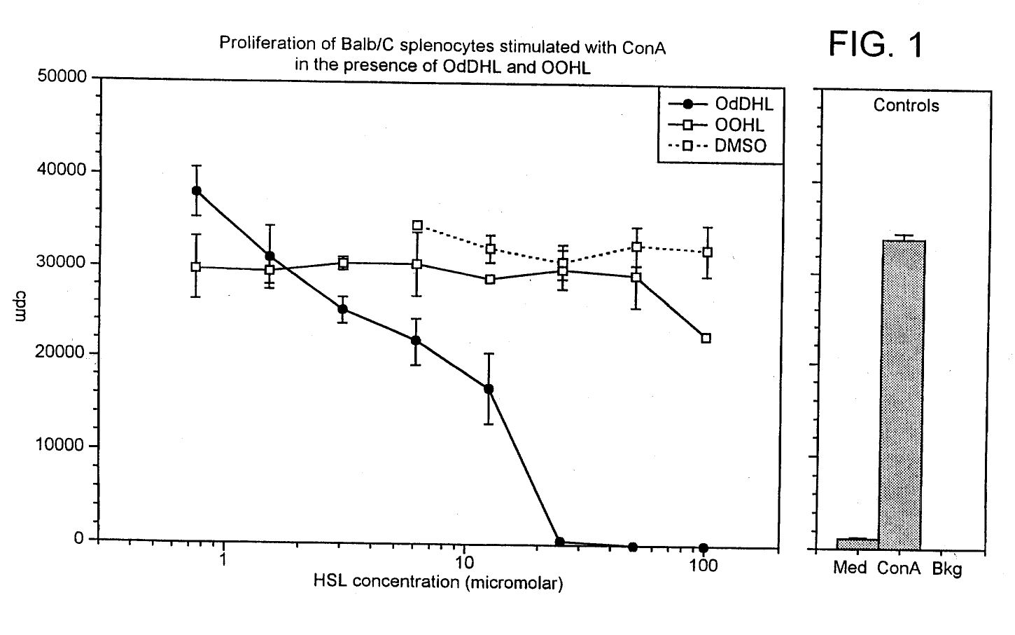

Mitogen (Concanavalin A) induced murine splenocyte proliferation was indicated by the incorporation of tritated thymidine into the DNA in the mouse spleen cells as shown by counts per minute using the scintillation counter. The inhibitory effect of an HSL compound being tested on cell proliferation was indicated by a reduction in counts per minute. FIG. 1 shows the plots of counts per minute (cpm) against the concentrations (micromolar) of the HSL compounds N-(3-oxododecanoyl)-L-homoserine lactone (OdDHL) and N-(3-oxooctanoyl)-L-homoserine lactone (OOHL) and the vehicle dimethylsulphoxide (DMSO). It can be seen, from this figure, that OdDHL inhibits splenocyte proliferation. In contrast, OOHL and DMSO failed to inhibit proliferation.

The IC50 value, i.e., the concentration (micromolar) of a compound which inhibits cell proliferation thymidine incorporation by 50% was determined for several compounds of the present invention and these IC 50 values are shown in column A of the Table below.

II. ConA Mitogen-Stimulated Proliferation of Human PBMC

Blood specimens were obtained with consent from healthy human volunteers. Human peripheral blood mononuclear cells (PBMC) were isolated from heparinised whole blood by buoyant density centrifugation over Histopaque 1077 (Sigma, Poole, UK) at 600 g for 20 minutes. PBMC harvested from the ‘buffy’ layers were washed twice with RPMI 1640 medium and resuspended in CTCM. HSL compounds were tested at similar dilutions as for murine splenocytes in 200 μl of CTCM, containing 1 μg/ml of ConA and 100,000 PBMC. Human PBMC were incubated for 48 h at 37° C. in 5% CO₂-air, followed by pulsing with 0.25 μCi [³H]-thymidine (see above). After a further incubation of 24 h cells were harvested onto fibreglass filters and then counted in the presence of MicroScint-O with the Packard TopCount.

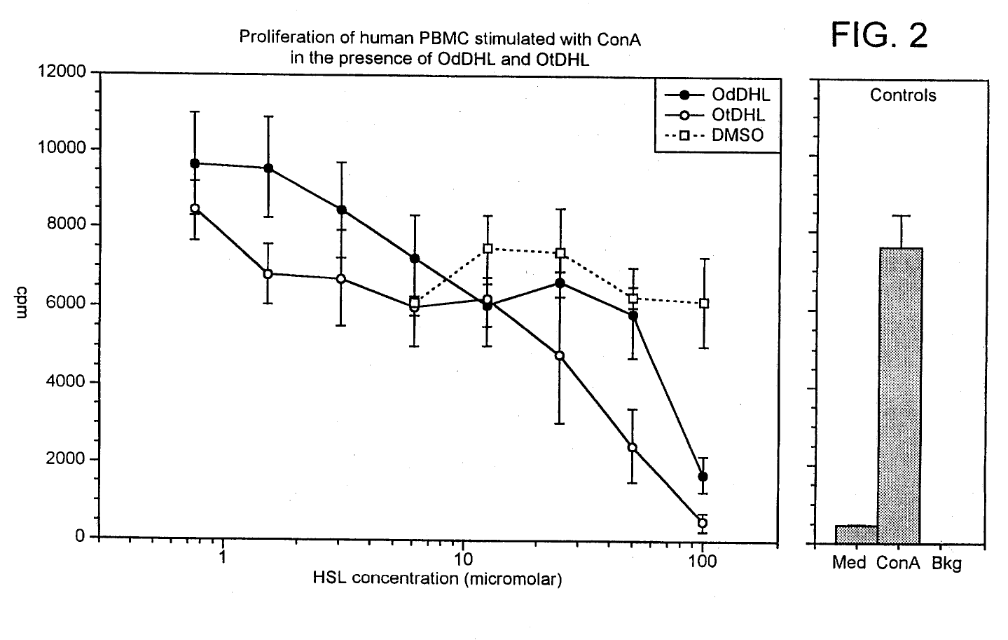

Concanavalin induced cell proliferation of human peripheral blood mononuclear cells (PBMC) was tracked, as described in I above, by a measurement of counts per minute using the scintillation counter. The inhibitory effect of an HSL compound being tested on cell proliferation was indicated by a reduction in counts per minute. FIG. 2 shows the plots of cpm against the concentrations of OdDHL, N-(3-oxotetradecanoyl)-L-homoserine lactone (OtDHL) and DMSO (vehicle). As can be seen, both OdDHL and OtDHL inhibited proliferation of human PBMC stimulated with Concanavalin A.

The IC50 values for several HSL compounds of the invention were determined and these are shown in columns B, C and D in the Table below. Columns B, C and D represent different sources of human PBMC samples used.

III. TNF-alpha Production from LPS-Stimulated Human PBMC

Bacterial lipopolysaccharide (LPS) stimulates the production of a variety of cytokines, including TNF-alpha, from human PBMC; these cytokines in turn influence the development of T cells, supporting a T helper 1 conducive milieu. Human PBMC prepared from whole blood by buoyant density centrifugation were resuspended in CTCM. HSL compounds were again tested at similar dilutions as for murine splenocytes in 200 μl of CTCM, containing 5×10⁻⁵ μg/ml LPS Escherchia coli strain 055:B5 (Sigma, Poole, UK) and 100,000 PBMC. Following incubation for 24 h at 37° C. in 5% CO₂-air, the cell culture supernatants were collected and tested for TNF-alpha levels by ‘sandwich’ ELISA. Briefly, 96-well Nunc MaxiSorp (Life Technologies, Paisley, UK) plates were coated with 50 μl of a 2 μg/ml solution of mouse anti-human TNF-alpha monoclonal antibody (Pharmingen, UK) in 0.05 M carbonate/bicarbonate buffer, pH 9.6 overnight at 4° C. After washing the plates three times with PBS-Tween, which contained phosphate buffered saline (PBS) with 0.5% (vol/vol) Tween-20 (Sigma, Poole, UK), the plates were blocked with 1% (wt/vol) bovine serum albumin (BSA) (Sigma, Poole, UK) at room temperature for 2 h. Following three washes with PBS-Tween, 50 μl of cell culture supernatants were added and incubated overnight at 4° C.; standard human TNF-alpha (Pharmingen, UK) ranging from 2000 to 31.25 pg/ml were included for each plate. After four washes with PBS-Tween, 50 μl of a second antibody, biotinylated mouse anti-human TNF-alpha monoclonal antibody (Pharmingen, UK) was added at 0.5 μg/ml diluted in 1% BSA in PBS-Tween and incubated at room temperature for 1 h. Following four washes, the bound biotinylated antibody was detected with 50 μl of a 1:1,000 dilution of Streptavidin-peroxidase (Pharmingen, UK). At the end of an hour incubation at room temperature, the plates were thoroughly washed six times with PBS-Tween and the assay was developed by the addition of 100 μl of 0.1 mg/ml of tetramethyl benzidine subtrate (Sigma, Poole, UK) in 0.1 M sodium acetate buffer, pH 6 containing 0.03% H₂O₂. The enzyme reaction was stopped with 50 μl of 2.5 M H₂SO₄ after an incubation of 10 minutes at room temperature and the development was read at 450 nm with a spectrophotometric 96-well plate reader (Dynex).

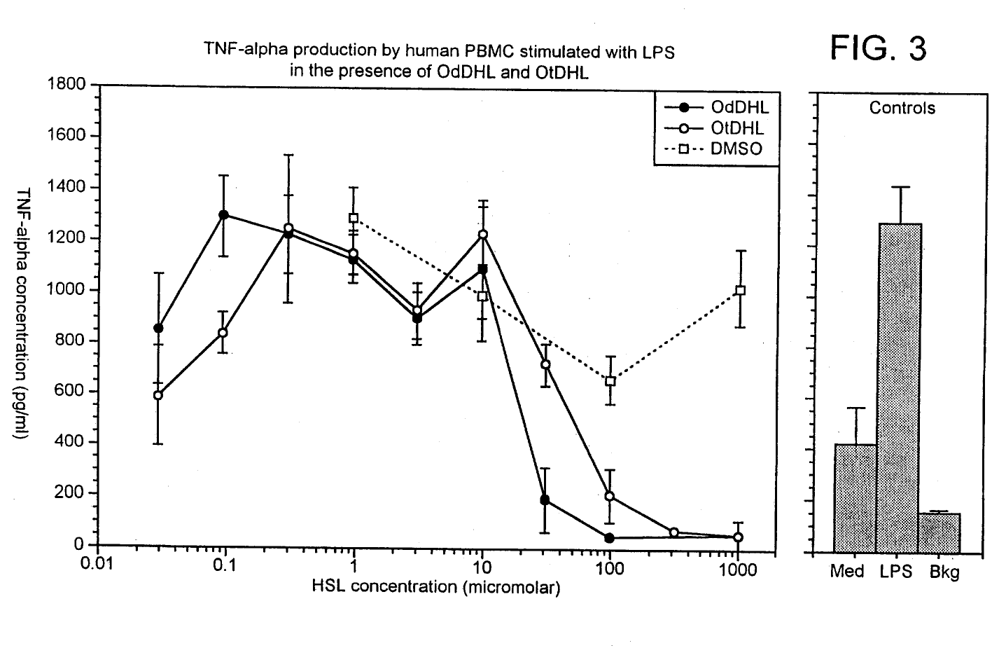

The effect of the concentration of certain HSL compounds of the invention on LPS induced TNF-α production by human PBMC was observed. FIG. 3 shows plots of TNF-α concentrations (pg/ml) against the concentration (micromolar) of OdDHL, OtDHL and DMSO (vehicle). As can be seen, both OdDHL and OtDHL inhibited the secretion of the T helper 1-supporting cytokine TNF-α. The IC50 values, i.e., the concentration (micromolar) of a compound which inhibits TNF-α secretion by 50%, was determined for some of the HSL compounds of the invention and these are shown in column E in the Table below.

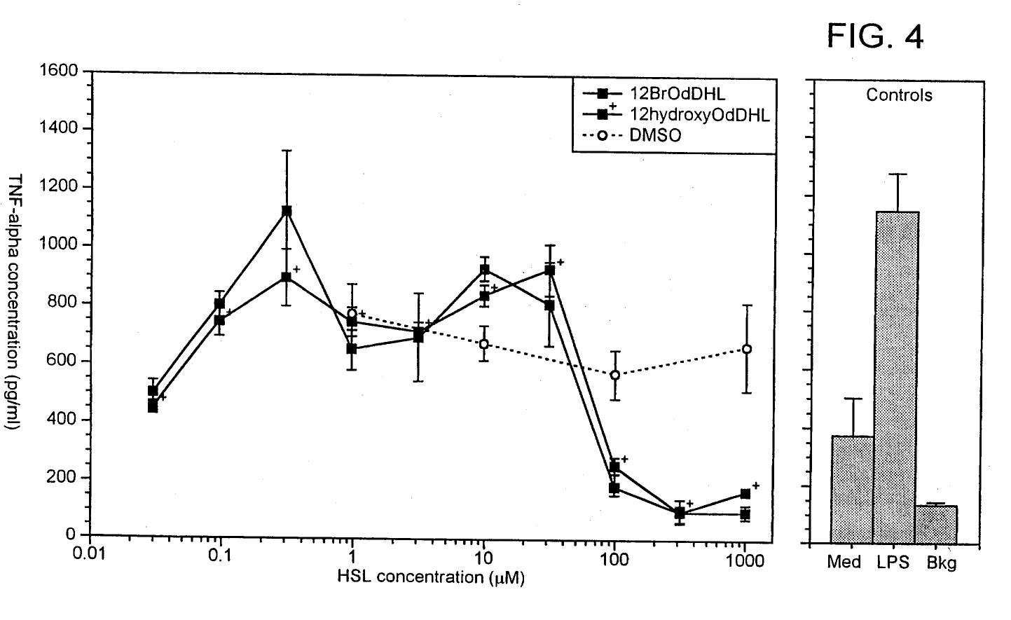

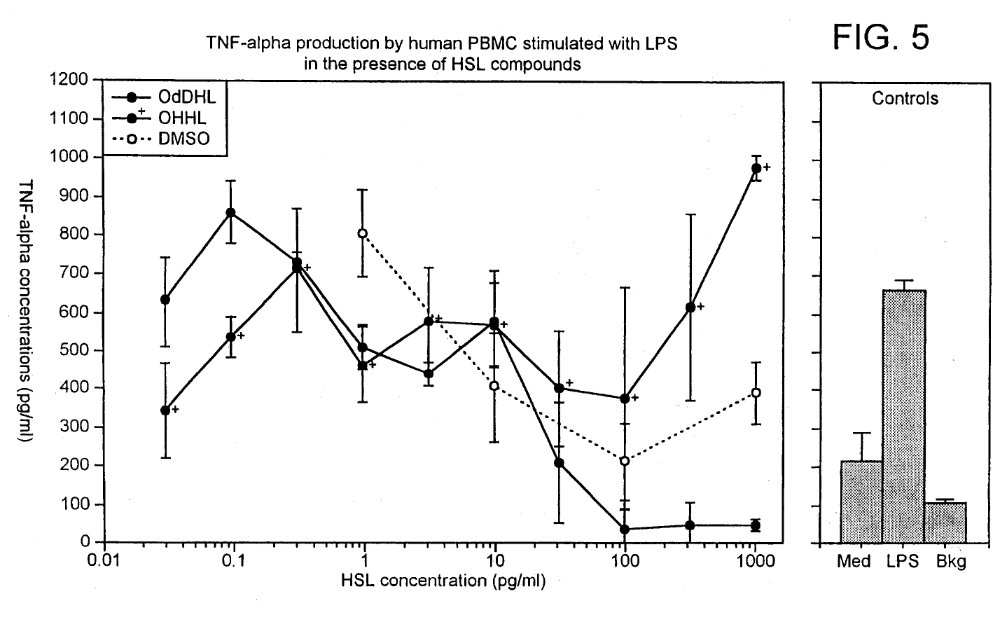

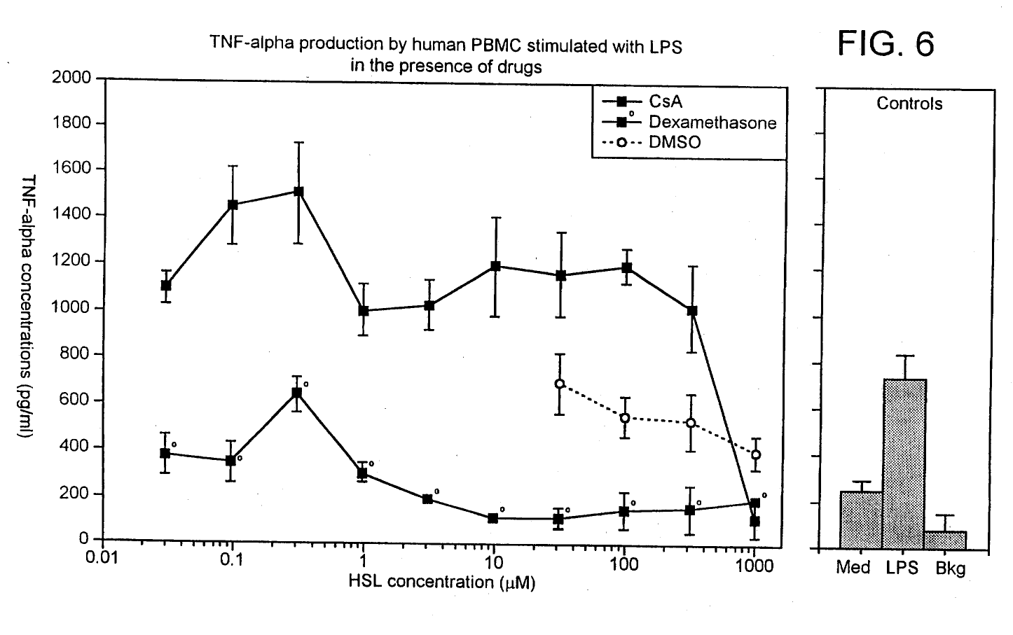

Similar studies were carried out using, as the HSL compounds, N-(12-bromo-3-oxododecanoyl)-L-homoserine lactone (12BrOdDHL) and N-(12-hydroxy-3-oxododecanoyl)-L-homoserine lactone (12hydroxyOdDHL) and the plots for these are shown in FIG. 4. For comparison purposes, similar studies were carried out using, as the HSL, the known shorter side chain compound N-(3-oxohexanoyl)-L-homoserine lactone (OHHL) and the plot for this is shown in FIG. 5. The difference in activity between OHHL and OdDHL is marked. Also for comparison purposes, similar studies were carried out using the known drugs dexamethasone and Cyclosporin A (CsA) and the plots for these are shown in FIG. 6. The IC50 value for dexamethasone was determined to be 500.

IV. Optimisation of Cell Culture Conditions

In the cell culture assays the number of cells used (mouse splenocytes and human PBMC) was initially optimised to 100,000 cells per well. The optimal dose of ConA of 1 μg/ml used in the cell proliferation assays was determined from ConA titration curves. A similar titration curve was established for LPS stimulation to obtain an LPS concentration which stimulated a suboptimal level of TNF-alpha release from human PBMC.

| EX | | NAME | | | | | |
|---|---|---|---|---|---|---|---|
| NO. | COMPOUND TESTED | ABBREVIATION | A | B | C | D | E |
| 1 | N-(3-Oxoundecanoyl)-L-homoserine lactone | OuDHL | 9 | 63 | 13 | 44 | |
| 2 | N-(11-Bromo-3-oxoundecanoyl)-L-homoserine lactone | 11BrOuDHL | 6 | 18 | | | |
| 3 | N-(10-Methyl-3-oxoundecanoyl)-L-homoserine lactone | 10MeOuDHL | 8 | | | 25 | |
| 4 | N-(10-Methoxycarbonyl-3-oxodecanoyl)-L-homoserine lactone | 10(MeO₂C)ODHL | 73 | 20 | | 63 | |
| 5 | N-(6-Methyl-3-oxoundecanoyl)-L-homoserine lactone | 6MeOuDHL | 5 | 20 | | | |
| 6 | N-(3-Oxododecanoyl)-L-homoserine lactone | OdDHL | 4 | 60 | 7 | 34 | 17 |
| 7 | N-(12-Bromo-3-oxododecanoyl)-L-homoserine lactone | 12BrOdDHL | 6 | 20 | | 7 | 63 |
| 8 | N-(3-Oxotridecanoyl)-L-homoserine lactone | OtriDHL | 4 | 41 | 7 | 25 | |
| 9 | N-(13-Bromo-3-oxotridecanoyl)-L-homoserine lactone | 13BrOtriDHL | 3 | 8 | | 12 | |
| 10 | N-(3-Oxo-12-tridecenoyl)-L-homoserine lactone | 12dbOtriDHL | 7 | | | | |
| 11 | N-(3-Oxotetradecanoyl)-L-homoserine lactone | OtDHL | 6 | 32 | 12 | 30 | 35 |
| 12 | N-(14-Bromo-3-oxotetradecanoyl)-L-homoserine lactone | 14BrOtDHL | 8 | | | | |
| 13 | N-(13-Methoxycarbonyl-3-oxotridecanoyl)-L-homoserine lactone | 13(MeO₂C)OtriDHL | 6 | 15 | | 20 | |
| 14 | N-(3-Oxo-7-tetradecenoyl)-L-homoserine lactone | 7cisOtDHL | 17 | | | | |
| 15 | N-(3-Oxo-9-tetradecenoyl)-L-homoserine lactone | 9cisOtDHL | | | | | |
| 16 | N-(3-Oxo-10-tetradecenoyl)-L-homoserine lactone | 10cisOtDHL | 15 | | | | |
| 17 | N-(3-Oxo-11-tetradecenoyl)-L-homoserine lactone | 11cisOtDHL | 12 | | | | |
| 18 | N-(3-Oxo-13-tetradecenoyl)-L-homoserine lactone | 13dbOtOHL | 10 | | | | |
| 19 | N-(12-Hydroxy-3-oxododecanoyl)-L-homoserine lactone | 12hydroxyOdOHL | 9 | | | | 70 |
| 20 | N-[11-(N,N-Dimethylcarbamoyl)-3-oxoundecanoyl]-L-homoserine lactone | 11(Me₂NCO)OuDH L | 35 | | | | |
| 21 | N-(3-Hydroxydodecanoyl)-L-homoserine lactone | HdDHL | 12 | | | | |
| 22 | N-(3-Hydroxytetradecanoyl)-L-homoserine lactone | HtDHL | 7 | | | | |
| 23 | N-(3-Hydroxy-7-tetradecenoyl)-L-homoserine lactone | 7cisHtDHL | 12 | | | | |
| 24 | N-(3-Hydroxy-9-tetradecenoyl)-L-homoserine lactone | 9cisHtDHL | 15 | | | | |
| 25 | N-(3-Hydroxy-10-tetradecenoyl)-L-homoserine lactone | 10cisHtDHL | 20 | | | | |
| 26 | N-(3-Hydroxy-11-tetradecenoyl)-L-homoserine lactone | 11cisHtDHL | 20 | | | | |
| 27 | N-(3-Hydroxy-13-tetradecenoyl)-L-homoserine lactone | 13cisHtDHL | 18 | | | | |

Notes to Table
Column A shows IC50 values (μM) for compounds in inhibition of ConA induced murine splenocyte proliferation (Experiment I)
Columns B, C & D show, for different sources of human PBMC, IC50 values (μM) for compounds in inhibition of ConA induced cell proliferation of PBMC (Experiment II)
Column E shows IC50 values (μM) for compounds in inhibition of LPS induced production of TNF-α by human PBMC (Experiment III)

1. A compound of the formula I 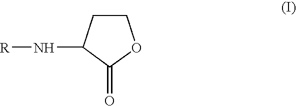
in which R is an acyl group of the formula 11 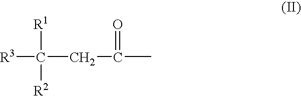
wherein one of R¹ and R² is H and the other is selected from OR⁴, SR⁴ and NHR⁴, wherein R⁴ is H or 1-6C alkyl, or R¹ and R² together with the carbon atom to which they are joined form a keto group, and R³ is a straight or branched chain, saturated or unsaturated aliphatic hydrocarbyl group containing from 8 to 11 carbon atoms and is optionally substituted by one or more substituent groups selected from halo, 1-6C alkoxy, carboxy, 1-6C alkoxycarbonyl, carbamoyl optionally mono- or disubstituted at the N atom by 1-6C alkyl and NR⁵R⁶ wherein each of R⁵ and R⁶ is selected from H and 1-6C alkyl or R⁵ and R⁶ together with the N atom form a morpholino or piperazino group, or any enantiomer thereof, with the proviso that R is not a 3-oxododecanoyl group.

2. A compound according to claim 1, wherein the R group is selected from 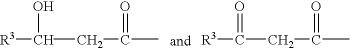
wherein R³ is as defined in claim 1.

3. A compound according to either claim 1 or claim 2, wherein the group R³ is an 8-11C straight or branched chain alkyl group optionally substituted by a substituent selected from bromo, carboxy and methoxycarbonyl.

4. A compound according to claim 3, wherein the R³ group is such that the group R in formula I is selected from;
3-oxoundecanoyl;
11-bromo-3-oxoundecanoyl;
10-methyl-3-oxoundecanoyl;
6-methyl-3-oxoundecanoyl;
3-hydroxydodecanoyl;
12-bromo-3-oxododecanoyl;
3-oxotridecanoyl;
13-bromo-3-oxotridecanoyl;
3-hydroxytetradecanoyl;
3-oxotetradecanoyl;
14-bromo-3-oxotetradecanoyl; and
13-methoxycarbonyl-3-oxotridecanoyl.

5. A compound according to either claim 1 or claim 2, wherein the group R³ is an 8-11C straight or branched chain alkenyl group optionally substituted by a substituent selected from bromo, carboxy and methoxycarbonyl.

6. A compound according to claim 5, wherein the R³ group is such that the group R in formula I is selected from;
3-oxo-12-tridecenoyl;
3-oxo-7-tetradecenoyl
3-hydroxy-7-tetradecenoyl;
3-oxo-9-tetradecenoyl;
3-hydroxy-9-tetradecenoyl;
3-oxo-10-tetradecenoyl;
3-hydroxy-10-tetradecenoyl;
3-oxo-11-tetradecenoyl;
3-hydroxy-11-tetradecenoyl;
3-oxo-13-tetradecenoyl; and
3-hydroxy-13-tetradecenoyl.

7. A pharmaceutical composition comprising a therapeutically-effective amount of a compound of any one of claims 1 to 6, or an enantiomer thereof, together with at least one pharmaceutically-acceptable carrier or diluent.

8. The use of a compound according to any one of claims 1 to 6, or an enantiomer thereof, for the manufacture of a medicament for the treatment of a disease of a living animal body, including a human, which disease is responsive to the activity of an immunosuppressant.

9. The use of a compound according to any one of claims 1 to 6, or an enantiomer thereof, for the treatment of an autoimmune disease in a living animal body, including a human.

10. The use according to claim 9, wherein the autoimmune disease is selected from psoriasis, multiple sclerosis and rheumatoid arthritis.

11. A method of treating a disease of a living animal body, including a human, which disease is responsive to the activity of immunosuppressants which method comprises administering to the living animal body, including a human, a therapeutically-effective amount of a compound according to any one of claims 1 to 6 or an enantiomer thereof.

12. The use of N-(3-oxododecanoyl)homoserine lactone, including an enantiomer thereof, for the manufacture of a medicament for the treatment of psoriasis in a living animal body, including human.

13. A method of treating psoriasis in a living animal body, including human, which method comprises administering to the living animal body, including human, a therapeutically-effective amount of N-(3-oxododecanoyl)homoserine lactone or an enantiomer thereof.
